(19) United States
(12) Patent Application Publication
(10) Pub. No.: US 2019/0345430 A1
(43) Pub. Date: Nov. 14, 2019

(54) DEVICES AND METHODS FOR BIO-PROCESSING CELLULAR SAMPLES

(71) Applicant: CESCA THERAPETEUTICS, INC., Rancho Cordova, CA (US)

(72) Inventors: Dalip SETHI, Rancho Cordova, CA (US); Nicolas A. BRUQUE, Rancho Cordova, CA (US); Xiaochun XU, Rancho Cordova, CA (US)

(21) Appl. No.: 16/475,952

(22) PCT Filed: Jan. 8, 2018
(86) PCT No.: PCT/US18/12817

§ 371 Date: Jul. 3, 2019

Related U.S. Application Data
(60) Provisional application No. 62/443,785, filed on Jan. 8, 2017.

(51) Int. Cl. C12M 1/00; A61M 1/02; A01N 1/02; C12M 3/00

(52) U.S. Cl. CPC C12M 23/14; A61M 2202/0486; A61M 1/0272; A61M 1/025; A01N 1/0268; A01N 1/0284; C12M 23/26; C12M 23/46; C12M 43/00; C12M 39/00; A61M 2205/6072; A61M 2202/0429; A61M 2202/0462; A61M 2202/10; A61M 2202/0415; A61M 2202/07; A61M 1/0209

(57) ABSTRACT

The present disclosure relates to completely closed systems suitable for bio-processing of cellular samples, for example peripheral blood samples used for immunotherapy applications, and related methods of use. The systems are not open to the air, thus allowing for sterile sample processing and transfer of the sample throughout the entirety of bio-processing. Each component of the disclosed systems contains a unique identifier, allowing for traceability of the sample as it proceeds through the various steps involved in bio-processing. The identifier ultimately traces the sample back to the patient from which the sample was derived. Certain embodiments provide a unique freezing bag for long-term storage of cellular samples. The freezing bag has a unique identifier that allows for easy traceability and retrieval of a bio-archived sample and at least two ports, one for sample testing, another for sterile docking to a device that allows for delivery of its contents to a patient.

### BACKGROUND

Cellular bioprocessing is a form of biopharmaceutical manufacturing, the goal of which is to establish reproducible and robust manufacturing processes for the production of therapeutic cells.

Current cellular manufacturing processes are highly user dependent, requiring human intervention at numerous points. Because of that dependence, current processes are tedious, highly variable and expensive.

By way of example, most of the current manufacturing processes occur as follows: a cell sample (whole blood, bone marrow, cord blood, etc.) is obtained from a patient. The obtained biological sample is transferred to laboratory for bio-processing. At the very beginning of laboratory-based processing a cell separation technique, such as polysaccharide (Ficoll)-dependent cell separation, is done to obtain a desired cell fraction enriched in mononucleated cells (MNC) and reduced in red blood cell (RBC) concentration. The MNC enriched cell fraction is isolated and transferred to a bioreactor for cellular expansion. The expanded cellular product is then moved from the bioreactor where they are washed, to remove the excess media and cell debris, and concentrated. Test samples are removed from the washed and concentrated cell product for quality testing and, if the test results show an acceptable product, the final engineered cellular product is either prepared for long term storage, cryopreservation, or administered to the patient from whom the sample was derived, or both. As can be appreciated, several of these steps require moving the sample from one container to another, not only requiring user intervention, risk of mislabeling the sample during processing, but also potentially exposing the sample to the environment, thus putting sterility at risk. The potential for compromised sterility is unacceptable for patients, particularly those who are immunocompromised.

### SUMMARY

Thus, an optimized, closed cellular bioprocessing and manufacturing system is needed. Such a system is provided by the present disclosure. The disclosed system eliminates user dependency, resulting in substantially more hands-off time, thereby allowing optimization of expensive technical resources.

The devices, systems, and methods disclosed herein have several features, no single one of which is solely responsible for its desirable attributes. Without limiting the scope of the claims that follow, certain features of the disclosed devices, systems and methods will be discussed briefly. After considering this discussion, and particularly after reading the section entitled “Detailed Description” a person having ordinary skill in the art will understand how the features of the devices, systems and methods provide several advantages over traditional systems and methods.

In one aspect, a system for cellular bioprocessing and manufacturing is provided, the system comprising: one or more processing bagsets, a culture bagset and a washing bagset, wherein. Each bagset comprises the same 2D barcode that is unique to the system and each bagset is configured to be in fluid connection with the other bagsets via one or more luer connections or via one or more sterile docks using a sterile connection device.

In a first embodiment of the system, the one or more processing bagsets of the system comprise flexible components and the flexible components comprise a processing bag, a red blood cell bag, and a cell concentrate bag. The system is closed to the outside environment and the components are in fluid connection with each other via a plurality of tubes.

The processing bag is in fluid connection with the red blood cell bag via a first tube; the processing bag is in fluid connection with the cell concentrate bag via a second tube; and the red blood cell bag and the cell concentrate bag are not directly connected to each other.

Volume transfer between the components is controlled via a multi-port valve that is directly connected to each of the processing bag, the red blood cell bag, and the cell concentrate bag.

The flexible processing bagset is configured for single use and is disposable. The processing bag can be made from a material selected from ethylene vinyl acetate (EVA), poly(vinyl) chloride (PVC) and other plastics; the red blood cell bag can be made from a material selected from PVC or other plastics; and the cell concentrate bag can be made from EVA, PVC or other plastics.

The processing bag can comprise an inlet line at the top of the processing bag that is in fluid connection with the interior of the processing bag, wherein the inlet line comprises a sterile connection selected from a female luer connection and a sterile dock, and wherein the inlet line is configured for receipt of a sample from outside of the processing bagset.

The cell concentrate bag can comprise a large compartment, and a small compartment connected by two channels; and one or more ports configured for removal of the contents of the cell concentrate bag away from the processing bagset, wherein the one or more ports are selected from spike ports, luer connections and sterile docks.

The multiport valve comprises an outer portion having three connectors and an inner portion, the inner portion comprising a handle and barrel configured to move between a closed position, a first open position and a second open position.

The first open position permits fluid flow from the processing bag through the multiport valve to the red blood cell bag; and the second position permits fluid flow from the processing bag through multiport valve to the cell concentrate bag.

In a second embodiment of the system, the one or more processing bagsets comprise a combination of flexible and rigid components. The rigid components comprise a processing container and a red blood cell container, and the flexible components comprise a cell concentrate bag. The system is closed to the outside environment and the components are in fluid connection with each other via a plurality of tubes.

The processing container is in fluid connection with the red blood cell container via a first tube; the processing container is in fluid connection with the cell concentrate bag via a second tube; and the red blood cell container and the cell concentrate bag are not directly connected to each other.

Volume transfer between the components is controlled via a multi-port valve that is directly connected to each of the processing container, the red blood cell container, and the cell concentrate bag.

The processing bagset is configured for single use and is disposable. The processing container is made from a material selected from ethylene vinyl acetate (EVA), poly(vinyl) chloride (PVC) and other plastics; the red blood cell container is made from a material selected from PVC or other plastics; and the cell concentrate bag is made from EVA, PVC, or other plastics.

The processing container comprises an inlet line at the top of the processing container that is in fluid connection with the interior of the processing container, wherein the inlet line comprises a sterile connection selected from a female luer connection and a sterile dock, and wherein the inlet line is configured for receipt of a sample from outside of the processing bagset.

The cell concentrate bag comprises: a large compartment, and a small compartment connected by two channels; and one or more ports configured for removal of the contents of the cell concentrate bag away from the processing bagset, wherein the one or more ports are selected from spike ports, luer connections and sterile docks.

The multiport valve comprises an outer portion having three connectors and an inner portion, the inner portion comprising a handle and barrel configured to move between a closed position, a first open position and a second open position.

The first open position permits fluid flow from the processing container through the multiport valve to the red blood cell container; and the second position permits fluid flow from the processing container through multiport valve to the cell concentrate bag.

In a second aspect, the present disclosure provides a three-dimensional freezing bag, comprising: an interior chamber, comprising a large compartment and a small compartment, the compartments connected by two channels; a first port defining a fluid connection between the large compartment and the exterior of the freezing bag; a second port defining a fluid connection between the small compartment and the exterior of the freezing bag; and a unique 2D barcode label; wherein the freezing bag is constructed for long-term cryo storage.

The freezing bag conforms to the C252.72 standard, wherein: the internal volume of the storage bag is 25 mL, there are a total of 2 ports leading out of the interior chamber of the freezing bag, and the freezing bag has a thickness depth of 7.2 mm.

The large compartment has a total volume of 20 mL and the small compartment has a total volume of 5 mL.

The first port is configured to receive a cellular sample from outside of the freezing bag, and the first port is also configured to deliver the contents of the large chamber outside of the freezing bag.

The second port is configured to deliver at least some of the contents of the small compartment outside of the freezing bag.

The ports comprise sterile connections selected from luer connections and sterile docks for connection using a sterile connection device.

The freezing bag is rated for cryogenic preservation of cellular samples in liquid nitrogen and is made from a material selected from ethylene vinyl acetate (EVA), a polyolefin-EVA blend, a fluorinated ethylene propylene (FEP) material, and combinations of any of the foregoing.

The unique 2D barcode present on the freezing bag corresponds to a 1D barcode present on a cryogenic storage cassette.

In a third aspect, the present disclosure provides a method of producing and cryo storing an engineered autologous cellular product. In some embodiments, the method comprises: obtaining a cellular sample from a subject; transferring the sample to one or more cell processing bagsets without exposing the sample to the outside environment, by attaching male and female luer lock connectors between the container in which the sample is obtained and the one or more processing bagsets, or by sterile-docking the tubing of the container in which the sample was obtained and the one or more processing bagsets using a sterile connection device; placing the one or more processing bagsets in one or more processing containers; centrifuging the one or more processing containers, thereby stratifying and separating the cellular sample based on the density, size of the cells and starting volume; transferring the desired cellular concentrate via gravity flow from the one or more processing bagsets to a culture bag without exposing the sample to the outside environment, by attaching male and female luer lock connectors between the one or more processing bagsets and the culture bag, or by sterile-docking the tubing of the one or more processing bagsets and the tubing of the culture bag using a sterile connection device; incubating the culture bag, thereby expanding the desired cellular concentrate; transferring the expanded cellular concentrate via gravity flow from the culture bag to a washing bagset which, by attaching male and female luer lock connectors to each other between the culture bag and the washing bagset, or sterile-docking the tubing of the culture bag and the washing bagset using a sterile connection device; washing the expanded cellular concentrate, thereby separating cellular waste byproducts generated from expansion from an engineered cell product; transferring the engineered cell product from the washing bagset to a freezing bag by attaching male and female luer lock connectors to each other between the washing bagset and the freezing bag, or sterile-docking the tubing of the washing bagset and the freezing bag using a sterile connection device, wherein the transfer occurs via centifugal force or gravity flow; transferring the freezing bag into a cryo-freezing overwrap bag and canister; and transferring the freezing bag, cryo-freezing overwrap and canister into a controlled rate cryo-freezing system that uses liquid nitrogen vapor to freeze the engineered cell product and maintain it in a cryo-preserved state.

The cellular sample can be selected from peripheral blood, whole blood, bone marrow, cord blood, and combinations of any of the foregoing.

The one or more cell processing bagsets, the culture bagset, the washing bagset and the freezing bag: are all configured for single use; are disposable; and all comprise the same unique 2D barcode that is specific to the sample.

In some embodiments, the method comprises, prior to centrifugation, depleting red blood cells from the sample.

In some embodiments, the sample is peripheral blood, red blood cells are depleted from the sample prior to centrifugation, and the centrifugation separates the peripheral blood into red blood cells, stem cell fraction and plasma.

In some embodiments, prior to the incubation of the culture bag, the method comprises supplementing a cellular growth media contained within the culture bag with one or more additives selected from cytokines, glucose and both.

The freezing bag is compliant with C252.72 standards, having a 25 milliliter (mL) storage volume, 2 ports or pig tails, and a depth of 7.2 mm.

The canister comprises a unique 1D barcode that is coupled to the 2D barcode on the freezing bag.

In some embodiments, the method further comprises, during the transfer of the freezing bag, cryo-freezing overwrap and canister into a controlled rate cryo-freezing system, scanning the 1D barcode to confirm the coupling of the 1D barcode information to the 2D barcode information.

In some embodiments, the method further comprises, after the transfer to the cryo-freezing system, transferring the freezing bag, cryo-freezing overwrap and canister to a storage location in a flask filled with liquid nitrogen for long-term cryo-storage.

### BRIEF DESCRIPTION OF THE DRAWINGS

The features and advantages of the present disclosure may be further explained by reference to the following detailed description and accompanying drawings that set forth illustrative embodiments.

FIG. 1 provides an overview of a method of using kit components for complete processing, cryostorage and transport of a biological sample, as provided by the present disclosure.

FIG. 2A depicts a representative example of a flexible cell processing bagset suitable for use in the systems and methods provided by the present disclosure. The bagset contains a unique identifier in the form of a 2D barcode, printed on each component.

FIG. 2B depicts a representative example of a semi-rigid cell processing bagset suitable for use in the systems and methods provided by the present disclosure. The bagset contains a unique identifier in the form of a 2D barcode, printed on each component.

FIG. 3 depicts a representative example of a cell culture bag having three ports and connectors. The cell culture bag contains a unique identifier in the form of a 2D barcode.

FIG. 4A depicts a representative example of a flexible cell washing bagset suitable for use in the systems and methods provided by the present disclosure. The bagset contains a unique identifier in the form of a 2D barcode, printed on each component.

FIG. 4B depicts a representative example of semi-rigid cell washing bagset suitable for use in the systems and methods provided by the present disclosure. The bagset contains a unique identifier in the form of a 2D barcode, printed on each component.

FIG. 5 depicts a representative example of a freezing bag suitable for use in the systems and methods provided by the present disclosure. The depicted dimensions are approximate and may not be to scale. The freezing bag contains a unique identifier in the form of a 2D barcode. In the depicted embodiment, the 2D barcode of the freezing bag corresponds to 1D barcode on the canister, as described herein.

### DETAILED DESCRIPTION

Before the embodiments of the disclosure are described, it is to be understood that such embodiments are provided by way of example only, and that various alternatives to the embodiments of the disclosure described herein may be employed in practicing the disclosure. Numerous variations, changes, and substitutions will now occur to those skilled in the art without departing from the disclosure.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. In case of conflict, the patent specification, including definitions, will control. In addition, the materials, methods, and examples are illustrative only and not intended to be limiting. Numerous variations, changes, and substitutions will occur to those skilled in the art without departing from the disclosure.

As used in the specification and claims, the singular forms “a”, “an” and “the” include plural references unless the context clearly dictates otherwise. For example, the term “a cell” includes a plurality of cells, including mixtures thereof.

In one aspect, the present disclosure provides completely closed systems suitable for bio-processing of cellular samples, for example peripheral blood samples used for immunotherapy applications. The systems are not open to the air, thus allowing for sterile sample processing and transfer of the sample throughout the entirety of bio-processing. Each of the disclosed systems contains a unique identifier, each component of the disclosed systems having the same unique identifier, allowing for traceability of the sample as it proceeds through bio-processing and, ultimately, allowing an expanded cellular sample to be traced back to the patient from which it was derived.

The systems provided by the present disclosure provide all of the components, from devices to methods, required for an easy-to-use cellular bio-processing and manufacturing process. Such components include, without limitation, one or more processing bagsets, a culture bagset and a washing bag-set. The washing bagset includes, among other things, a specially designed freezing bag. In some embodiments, the system is referred to as a CXP Platform.

The disclosed systems provide a sterile, closed environment in which a bio-processing cellular manufacturing process can take place. Put another way, the present disclosure provides a complete kit of devices and related methods for the bio-processing of cellular samples such as, for example, peripheral blood for immunotherapy applications. The systems allow for sterile processing and transfer of a cellular sample throughout the manufacturing process.

Even though some of the components of the disclosed systems are separable from each other, as described in detail below, they are configured to connect to each other in a completely sterile manner, thereby allowing transfer of the sample from one component to another during the manufacturing process. As noted above, each system contains a unique identifier that distinguishes it from another system, with each component of a single system containing the same unique identifier. The disclosed systems thus provide traceability at each step of the process, thereby reducing contamination and also reducing, if not eliminating, the risk that the manufactured cellular sample will be administered to the wrong subject.

In some embodiments, and as described in greater detail below, the unique identifier given to a single system is a unique, 2D barcode. The unique, 2D barcode is specific to a single system and each component of the system contains the same unique, 2D barcode. More specifically, for a single system, a unique, 2D barcode is imprinted onto each of the following system components: the processing bag-set, the culture bag-set and the washing bag-set, which includes, among other things, a specially designed freezing bag-set that will also have the same unique, 2D barcode.

In a second aspect, methods of using the closed systems are provided.

In a third aspect, the present disclosure provides a unique freezing bag. The freezing bag is a component of the disclosed systems and is suitable for use with the disclosed methods of bio-processing cellular samples. Like all of the components of the disclosed systems, the freezing bag has a unique identifier that allows for easy traceability and retrieval of a bio-archived sample. The freezing bag also has at least two ports, one for sample testing, another for sterile docking to a device that allows for delivery of its contents to a patient.

In some embodiments, the freezing bag is imprinted with a unique identifier that identifies the freezer bag as a component of a unique bioprocessing system. The unique identifier can be a unique, 2D barcode, which will match the unique, 2D barcode of the other components of the freezer bag's system. In some embodiments, and as described in detail below, the freezer bag's unique, 2D barcode matches a 1D barcode that is applied to a storage canister. The pairing of the 2D barcode placed on the freezer bag with the 1D barcode placed on the storage canister allows for easy traceability and retrieval when the manufactured cellular sample is stored in a bioarchive system.

In various embodiments, the freezer bag has, among other things, at least two ports, or pig tails. One such port or pigtail is used for sample quality testing or other testing as needed. The other port or pigtail is used for sterile docking to a device that delivers the cellular sample to the patient, for example a saline bag.

In various embodiments, the freezer bag is compliant with C_{252.72} standards. That is, the storage bag has a 25 milliliter (mL) storage volume, 2 ports or pig tails, and a depth of 7.2 mm.

### Methods of Use

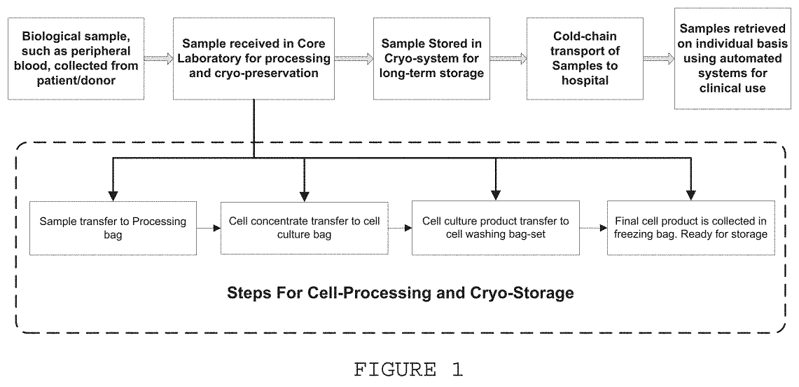

FIG. 1 presents an overview of one embodiment of a method of using the systems provided by the present disclosure. The method begins with the step depicted in the upper left-hand block and proceeds in the direction of the arrows provided for each step. The depicted method relates to the use of a disclosed system to process, cryo store and transport an autologous cellular product. In the depicted embodiment, the system comprises a set, or kit, of bagset disposables that collectively comprise a system provided by the present disclosure, which in some embodiments is a CXP device, and a Bioarchive system for long term storage of a manufactured cellular product.

The process begins by extracting a cellular sample, such as peripheral blood, whole blood, bone marrow, cord blood, etc., from a patient or donor (upper left hand box).

The sample is received and processed by a core laboratory equipped with a system encompassed by the present disclosure. The system comprises a plurality of disposable components including, without limitation, a sample processing bagset, a culture bagset, a washing bagset and a freezing bag, all tagged with the same unique and common identifier in the form of a 2D barcode printed on each component.

To process the sample for cryo-storage, the laboratory first transfers the sample to an appropriate number of cell processing bag sets based on the initial volume extracted from the patient. In some embodiments, each processing bagset used for the processing of this sample is a component of its own unique system and thus each processing bagset is imprinted with its own unique identifying 2D barcode. In that respect, if a single cellular sample is split up among multiple processing bagsets, each bagset will be processed via its own unique system. In other embodiments, each processing bagset used for the processing of this single sample is a component of the same system and thus each processing bagset is imprinted with the same unique identifying 2D barcode. In that respect, the entirety of the single sample is processed in a single system.

In some embodiments, the sample is then sterilely transferred from the one or more processing bagsets to a processing container. The sterile transfer is performed without exposing the sample to the outside environment. In that respect, the transfer can occur via any number of ways that will maintain the closed, sterile environment of the disclosed system(s). The transfer may be accomplished by attaching male and female luer lock connectors to each other between the one or more processing bagset(s) and the processing container, or sterile-docking the tubing of the one or more processing bagsets and the processing container using a sterile connection device. The sample is transferred by gravity flow from the one or more processing bags to the processing container, which in some embodiments is a portable CXP electromechanical processing device, for centrifugation. In other embodiments, the one or more processing bagsets are placed into one or more processing containers. In some embodiments, the CXP processing device is programmed to deplete red blood cells from the sample before the processing bagsets are loaded into a centrifuge. During centrifugation, the processing device—or portable CXP electromechanical device—automatically stratifies and separates the cellular sample based on the density, size of the cells and starting volume. For example, if the sample is peripheral blood, the processing device first depletes red blood cells from the sample, then centrifuges the sample in order to separate the peripheral blood into red blood cells, stem cell fraction and plasma. In doing so, the desired cellular fraction from the sample will be concentrated and can be easily removed from the one or more processing bagsets.

The desired cellular concentrate is then moved from the one or more processing bags in the processing device to a culture bag. The transfer may be accomplished by attaching male and female luer lock connectors to each other between the one or more processing bagsets and the culture bag, or sterile-docking the tubing of the one or more processing bagsets and the culture bag using a sterile connection device. The sample is transferred by gravity flow from the one or more processing bags to the culture bag. In the culture bag, the media in which the desired cellular fraction resides is supplemented with one or more desired additives, for example cytokines, glucose and/or other agents, in order to achieve a specific cellular engineered treatment. The culture bag is then placed into an incubator, where the desired cellular fraction is allowed to expand under desirable growth conditions. Expansion can occur for any desired amount of time, provided that the media in which the desired cellular fraction resides is capable of supporting growth of the cells of the desired cellular fraction.

Once the sample has sufficiently been treated and/or expanded, it is transferred to a washing bagset which, in some embodiments, is an AutoXpress processing bag. As with the other sample transfers, this transfer may be accomplished by attaching male and female luer lock connectors to each other between the one or more processing bagsets and the culture bag, or sterile-docking the tubing of the culture bag and the washing bagset using a sterile connection device. The sample is transferred by gravity flow from the culture bag to the washing bagset. In the washing bagset, the desired expanded and/or treated cellular sample is washed in order to separate the cellular waste byproducts generated from the expansion/treatment phase from the final engineered cell product. In some embodiments, the washing bagset is known as the CXP device preset, which is configured to conduct cellular washing.

Upon completion of washing, the desired expanded and/or treated cellular fraction is in condition for administration to the patient and/or cryo storage. The washed and concentrated cellular sample is transferred to a freezing bag. The sample may be collected in the desired freezing bag as part of the washing bagset, or as with the other sample transfers, this transfer may be accomplished by attaching male and female luer lock connectors to each other between the washing bagset and the freezing bag, or sterile-docking the tubing of the washing bagset and the freezing bag using a sterile connection device. The sample is transferred either by centifugal force or gravity flow from the washing bagset to the freezing bag.

The final washed cell product is collected into the freezing bag pursuant to C_{252.72} standards and a 2D barcode that matches the one or more processing bags, cell culture bagset and washing bagset. In those embodiments in which the freezer bag is compliant with C_{252.72} standards, the freezer bag has a 25 milliliter (mL) storage volume, 2 ports or pig tails, and a depth of 7.2 mm.

At this point, the method progresses past the “Steps For Cell-Processing and Cryo-Storage” and moves into long-term storage. The long-term storage can be, for example, a Bioarchive storage as disclosed in U.S. Pat. Nos. 5,964,095; 6,146,124; 6,232,115; 6,302,327; and/or 6,808,675.

After the desired expanded and/or treated cellular fraction is moved into the freezing bag, the freezing bag is loaded into a cryo-freezing overwrap bag and canister. In several embodiments, the canister includes a unique 1D barcode that is coupled, via a hardcopy or electrical database, to the 2D barcode on the freezing bag. This is done in order to ensure that the sample is traceable to the patient from whom it was derived. The combination of the freezing bag, cryo-freezing overwrap and canister is then loaded into a cryo-freezing system, which is a controlled rate freezing device that uses liquid nitrogen vapor to freeze the sample and maintain it in a cryo-preserved state.

In many embodiments, the cryo-freezing system/control rate freezer uses a validated freezing profile in order to ensure optimal cryo-preservation.

In order to ensure that the desired expanded and/or treated cellular fraction is linked with and/or delivered to the proper subject, during the controlled rate freezing process a robotic arm scans the 1D barcode present on the canister to confirm the information stored in the 1D barcode, which is coupled to the 2D barcode information present on each of the one or more processing bagsets, the culture bagset, the washing bag-set and the freezing bag. In some embodiments, at the end of the freezing process, a robotic arm removes the freezing bag, cryo-freezing overwrap and canister combination from the control rate freezer and transfers it to a storage location in a Dewar flask filled with liquid nitrogen for long-term cryo-storage.

When a health care provider, for example a hospital, is ready to administer the desired expanded and/or treated cellular fraction back into the subject from which it was derived, a user enters the 1D or 2D barcode information into the cryo-system for retrieval. The robotic arm receives that information, which is directly linked to the 1D barcode on the canister, and uses that information to automatically retrieve the sample from the liquid nitrogen for cold-chain transport to the subject.

Once the desired expanded and/or treated cellular fraction arrives at the location of the health care provider, it can either be stored again, for example in an in-house cryo-system, or administered to the subject.

### Systems

As stated above, the systems provided by the present disclosure provide all of the components required for an easy-to-use cellular bio-processing and manufacturing process. Such components include, without limitation, one or more processing bagsets, a culture bagset and a washing bagset. The washing bagset includes, among other things, a specially designed freezing bag.

### Processing Bagsets

The processing bagsets suitable for use with the systems and methods provided by the present disclosure can be flexible, or semi-rigid.

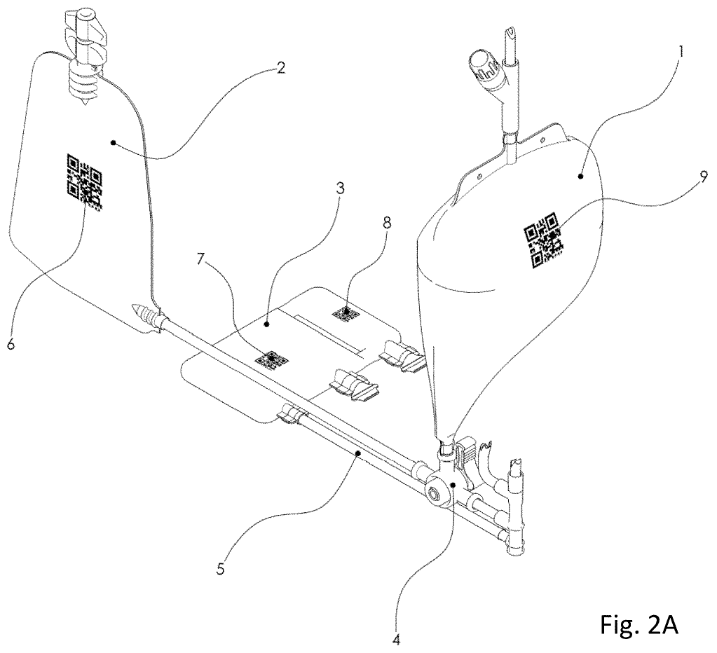

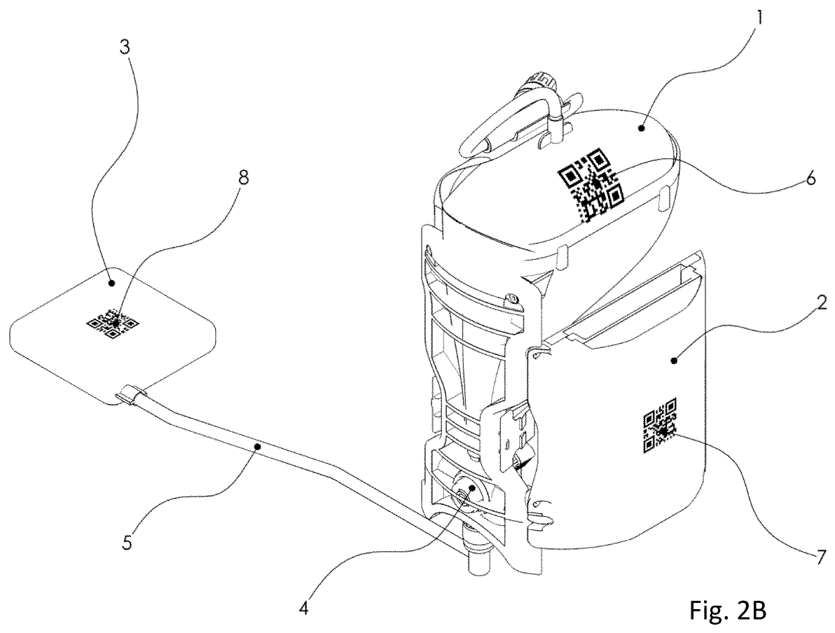

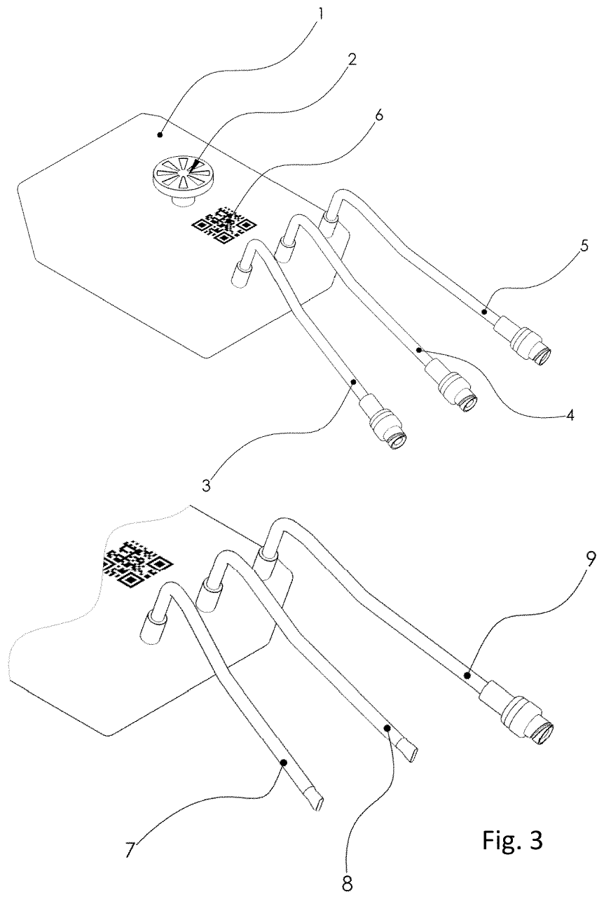

FIG. 2A depicts an example of one embodiment of a flexible processing bagset suitable for use with the systems provided by the present disclosure. The depicted flexible bagset comprises a plurality of components. In some embodiments, the components comprise: a processing bag (FIG. 2A-1), a red blood cell bag (FIG. 2A-2), and a cell concentrate bag (FIG. 2A-3). Volume transfer between the components is controlled via a multi-port valve (FIG. 2A-4). When a cellular sample is loaded into the disclosed systems, it is first loaded into the processing bag (FIG. 2A-1). The multiport valve (FIG. 2A-4) governs the flow of the sample between the other components of the flexible processing bagset.

As disclosed herein, each component of the flexible bag includes a unique 2D barcode label. In some embodiments, the 2D barcode is laser etched onto each component during processing in order to ensure traceability of the sample back to the subject from which it was derived. All of the 2D barcodes, (FIG. 2A-6), (FIG. 2A-7), (FIG. 2A-8) and (FIG. 2A-9) are identical.

The flexible processing bagset is closed to the outside environment and is disposable.

As noted above, the bagset comprises a processing bag (FIG. 2A-1), a red blood cell bag (FIG. 2A-2), and a cell concentrate bag (FIG. 2A-3), all of which are connected by lines or tubing to the multi-port valve (FIG. 2A-4), with inlet lines, clamps, filters, and sampling sites.

The processing bag (FIG. 2A-1) may be made of ethylene vinyl acetate (EVA), poly(vinyl) chloride (PVC) or other plastics. The red blood cell bag (FIG. 2A-2) may be made of PVC or other plastics. The cell concentrate bag (FIG. 2A-3) may be made of ethylene-vinyl acetate (EVA), although other plastics may be used.

Each of the three bags (FIG. 2A-1), (FIG. 2A-2) and (FIG. 2A-3) may be blow-molded. The red blood cell bag (FIG. 2A-2) may be radio frequency, high frequency or dielectric welded and may be blow-molded.

In some embodiments, the processing bag (FIG. 2A-1) is a three-dimensional bag having an asymmetric shape, including a top edge, curved side, straight side opposite the curved side, tapered bottom, and bottom outlet that is in fluid connection with the multiport valve (FIG. 2A-4). The top edge includes an inlet for receipt of the sample from a subject, as well as two holes that may be used to hang the processing bag in space. In other embodiments, the processing bag (FIG. 2A-1) may be shaped symmetrically such that its sides taper symmetrically towards bottom outlet.

The total volume of processing bag may be 25 up to 125 mL, although in several embodiments, when in use it is typically filled with about 50-150 mL of sample from a subject.

In order to receive a sample from a subject, the processing bag (FIG. 2A-1) is configured to receive an inlet line at a discrete point along the top line, which connects to an inlet that is in fluid connection with the interior of the processing bag (FIG. 2A-1). In some embodiments, the inlet line comprises a female luer connection which allows the processing bag set to be connected to a source of a cellular sample, be that a subject or a container containing the cellular sample to be transferred into processing bag (FIG. 2A-1). In other embodiments, the inlet line comprises a sterile dock for connection to a source of a cellular sample using a sterile connection device.

The red blood cell concentrate bag (FIG. 2A-2) may be a flat bag, having a top edge, bottom edge, and two substantially similar side edges. In some embodiments, the red blood cell bag includes a butterfly spike port along the top edge, which can be used to remove an aliquot of the red blood cells during bio-processing, should that be desired. The bottom edge includes an inlet at one corner that is in fluid connection with the processing bag, via the multiport valve (FIG. 2A-4).

The volume of red blood cell bag is up to 90 mL, although in use it is typically filled with about 30-80 mL.

The cell concentrate bag (FIG. 2A-3) is a three-dimensional bag that is rectangular in shape, having a top edge, bottom edge, and two compartments, a large compartment, and a small compartment—the large and small compartments connected by two channels. The top edge includes an inlet that is in fluid connection with the processing bag, via the multiport valve (FIG. 2A-4), and two ports that are used to remove the desired cellular fraction at the end of this step of the process. The ports may be spike ports, luer connections or sterile docks for connection using a sterile connection device.

The volume of the cell concentrate (FIG. 2A-3) bag is 10 to 50 mL, although in use, it is typically filled with about 25 mL, about 20 mL of which is present in the large compartment and about 5 mL of which is present in the small compartment.

The lines connecting each of the bags of the processing bagset are tubing that may be made of poly(vinyl) chloride (PVC), ethylene-vinyl acetate (EVA), or other materials.

In some embodiments, multiport valve (FIG. 2A-4) is a three-way stopcock in that has three connectors such that it can be connected to three bags: the processing bag (FIG. 2A-1), the red blood cell bag (FIG. 2A-2), and the cell concentrate bag (FIG. 2A-3). Other types of metering valves or stopcocks will also work, such as a four-way stopcock having four connectors.

In the depicted embodiment, the multiport valve (FIG. 2A-4) comprises an outer portion having three connectors and an inner portion. The outer portion may be made of polycarbonate. The inner portion includes a handle and barrel, integrally molded, which may be made of polyethylene. The barrel moves between several positions, including a closed position, defined as a position that does not allow any fluid flow through the multiport valve (FIG. 2A-4), and two open positions defined as positions that permit fluid flow through the multiport valve (FIG. 2A-4).

The two open positions include a first position that permits fluid flow from the processing bag (FIG. 2A-1) through the multiport valve (FIG. 2A-4) to the red blood cell bag (FIG. 2A-2) and a second position that permits fluid flow from the processing bag (FIG. 2A-1) through multiport valve (FIG. 2A-4) to the cell concentrate bag (FIG. 2A-3). All fluid flow through the bagset is accomplished via gravity.

FIG. 2B depicts a semi rigid embodiment of the processing bagset suitable for use with the systems provided by the present disclosure. In the depicted embodiment, the semi rigid processing bagset comprises a rigid (ridge) processing bag (FIG. 2B-1) or container, a rigid (ridge) red blood cell container (FIG. 2B-2) and a flexible cell concentrate bag (FIG. 2B-3) for processing a cellular sample. Volume transfer between the bags and container is controlled via a multi-port valve (FIG. 2B-4) when loaded into a CXP or CXP-II device. When a cellular sample is loaded into the disclosed systems, it is first loaded into the processing bag (FIG. 2B-1). The multiport valve (FIG. 2B-4) governs the flow of the sample between the other components of the semi rigid processing bagset.

As disclosed herein, each component of the semi rigid bag includes a unique 2D barcode label. In some embodiments, the 2D barcode is laser etched onto each component during processing in order to ensure traceability of the sample back to the subject from which it was derived. All of the 2D barcodes (FIG. 2B-6), (FIG. 2B-7) and (FIG. 2B-8) are identical.

The semi rigid processing bagset is closed to the outside environment and is disposable.

As noted above, the bagset comprises a processing bag (FIG. 2B-1), a red blood cell container (FIG. 2B-2), and a cell concentrate bag (FIG. 2B-3), all of which are connected by lines or tubing to the multi-port valve (FIG. 2B-4), with inlet lines, clamps, filters, and sampling sites.

The semi rigid bagset includes a rigid plastic housing for the processing bag (FIG. 2B-1) that maintains its shape and is capable of holding the entire bagset upright during operation. The housing is configured such that it contains a point of attachment for the red blood cell container, which also includes a rigid plastic backing that connects to the rigid housing. The cell concentrate bag is flexible and connected to the processing bag and red blood cell container via a line of tubing and the multiport valve (FIG. 2B-4).

The rigid plastic housing and rigid plastic backing can be made of any suitable rigid-plastic material. The rigid plastic material exhibits no elastic deformation, nor does it display any of the elastic behavior typically displayed by flexible plastics. The rigid plastic material made of any suitable rigid plastic including, for example, high-density polyethylene (HDPE) or polypropylene (PP).

Each of the rigid plastic housing and backing may be injection molded or die-cut.

The processing bag (FIG. 2B-1) is contained within the rigid plastic housing, which may or may not completely enclose the processing bag (FIG. 2B-1). In the depicted embodiment, the processing bag (FIG. 2B-1) is not completely enclosed within the rigid plastic housing; rather, the housing partially encloses the processing bag (FIG. 2B-1), holding it in place during use. The processing bag (FIG. 2B-1) may be made of ethylene vinyl acetate (EVA), poly(vinyl) chloride (PVC) or other plastics. The red blood cell container (FIG. 2B-2) may be made of PVC or other plastics and is attached to one side of a rigid plastic backing that is removably connected to the rigid plastic housing. The cell concentrate bag (FIG. 2B-3) may be made of ethylene-vinyl acetate (EVA), although other plastics may be used.

Each of the bags (FIG. 2B-1) and (FIG. 2B-3) and the container (FIG. 2B-2) may be blow-molded. The red blood cell container (FIG. 2B-2) may be radio frequency, high frequency or dielectric welded and may be blow-molded.

In some embodiments, the processing bag (FIG. 2B-1) is a three-dimensional bag having an asymmetric shape, including a top edge, curved side, straight side opposite the curved side, tapered bottom, and bottom outlet that is in fluid connection with the multiport valve (FIG. 2B-4). The top edge includes an inlet for receipt of the sample from a subject, as well as two holes that may be used to hang the processing bag in space. In such embodiments, the shape of the rigid plastic housing mirrors that of the processing bag (FIG. 2B-1), tapering from top to bottom, in order to properly house the processing bag (FIG. 2B-1). In other embodiments, the processing bag (FIG. 2B-1) may be shaped symmetrically such that its sides taper symmetrically towards bottom outlet. In such embodiments, the shape of the rigid plastic housing mirrors that of the processing bag (FIG. 2B-1), having symmetrical sides in order to properly house the processing bag (FIG. 2B-1).

The total volume of processing bag may be up to 125 mL, although in several embodiments, when in use it is typically filled with about 50-150 mL of sample from a subject.

In order to receive a sample from a subject, the processing bag (FIG. 2B-1) is configured to receive an inlet line at a discrete point along the top line, which connects to an inlet that is in fluid connection with the interior of the processing bag (FIG. 2B-1). In some embodiments, the inlet line comprises a female luer connection which allows the processing bag set to be connected to a source of a cellular sample, be that a subject or a container containing the cellular sample to be transferred into processing bag (FIG. 2B-1). In other embodiments, the inlet line comprises a sterile dock for connection to a source of a cellular sample using a sterile connection device.

The red blood cell concentrate container (FIG. 2B-2) may be a flat bag, having a top edge, bottom edge, and two substantially similar side edges. In some embodiments, the red blood cell bag includes a butterfly spike port along the top edge, which can be used to remove an aliquot of the red blood cells during bio-processing, should that be desired. The bottom edge includes an inlet at one corner that is in fluid connection with the processing bag, via the multiport valve (FIG. 2B-4). The container is mounted to the rigid plastic backing that is removably connected to the rigid plastic housing.

The volume of red blood cell container (FIG. 2B-2) is up to 90 mL, although in use it is typically filled with about 30-80 mL.

The cell concentrate bag (FIG. 2B-3) is a three-dimensional bag that is rectangular in shape, having a top edge and a bottom edge. In the depicted embodiment, the cell concentrate bag (FIG. 2B-3) comprises a single, large compartment with only a single opening that is in fluid connection with the other components via the multiport valve (FIG. 2B-4). In such embodiments, the cell concentrate bag (FIG. 2B-3) may be removed from the bagset via the connection to the multiport valve (FIG. 2B-4) via a luer connection or a sterile dock for connection using a sterile connection device, which allows the cell concentrate bag to be connected to other bagsets in a sterile manner, maintaining the cellular sample in a closed environment that is never exposed to the outside air.

In other embodiments, the cell concentrate bag (FIG. 2B-3) bag is a three-dimensional bag that is rectangular in shape, having a top edge and a bottom edge and two compartments, a large compartment, and a small compartment—the large and small compartments connected by two channels. The top edge includes an inlet that is in fluid connection with the processing bag, via the multiport valve (FIG. 2B-4), and two ports that are used to remove the desired cellular fraction at the end of this step of the process. The ports may be spike ports, luer connections or sterile docks for connection using a sterile connection device.

The volume of the cell processing bag (FIG. 2B-3) is 10 to 50 mL, although in use, it is typically filled with about 25 mL. In those embodiments in which the cell concentrate bag (FIG. 2B-3) has two compartments, the 25 mL capacity is divided such that about 20 mL of which is present in the large compartment and about 5 mL of which is present in the small compartment.

The lines connecting each of the bags of the processing bagset are tubing that may be made of poly(vinyl) chloride (PVC), ethylene-vinyl acetate (EVA), or other materials.

In some embodiments, multiport valve (FIG. 2B-4) is a three-way stopcock in that it has three connectors such that it can be connected to three bags: the processing bag (FIG. 2B-1), the red blood cell bag (FIG. 2B-2), and the cell concentrate bag (FIG. 2B-3). Other types of metering valves or stopcocks will also work, such as a four-way stopcock having four connectors.

In the depicted embodiment, the multiport valve (FIG. 2B-4) is contained within the rigid plastic housing and comprises an outer portion having three connectors and an inner portion. The outer portion may be made of polycarbonate. The inner portion includes a handle and barrel, integrally molded, which may be made of polyethylene. The barrel moves between several positions, including a closed position, defined as a position that does not allow any fluid flow through the multiport valve (FIG. 2B-4), and two open positions defined as positions that permit fluid flow through the multiport valve (FIG. 2B-4). In other embodiments, the rigid plastic housing may have an opening that is suitable to house the multiport valve (FIG. 2B-4) removably, such that the multiport valve (4) may be slid into and out of the housing.

The two open positions include a first position that permits fluid flow from the processing bag (FIG. 2B-1) through the multiport valve (FIG. 2B-4) to the red blood cell bag (FIG. 2B-2) and a second position that permits fluid flow from the processing bag (FIG. 2B-1) through multiport valve (FIG. 2B-4) to the cell concentrate bag (FIG. 2B-3). All fluid flow through the bagset is accomplished via gravity.

### Cell Culture Bagsets

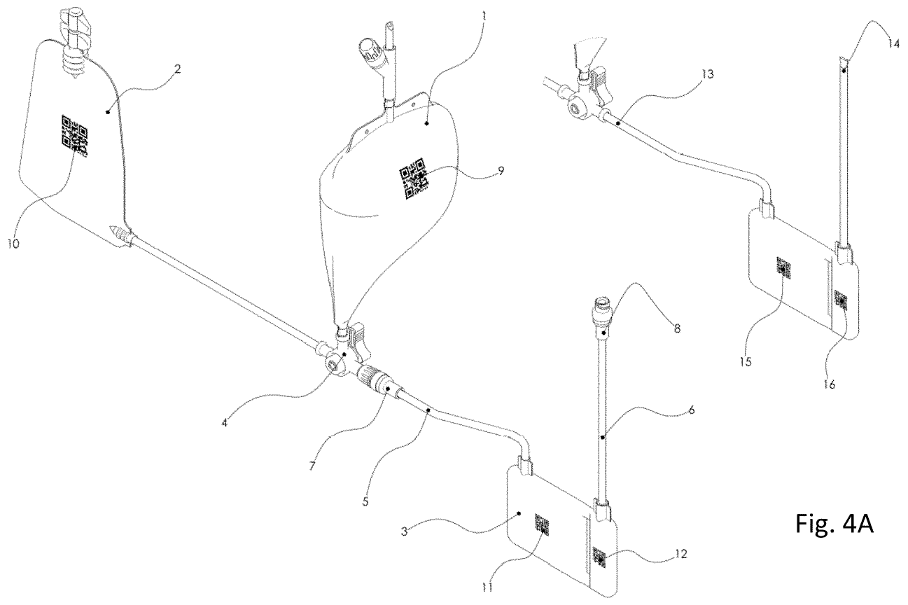

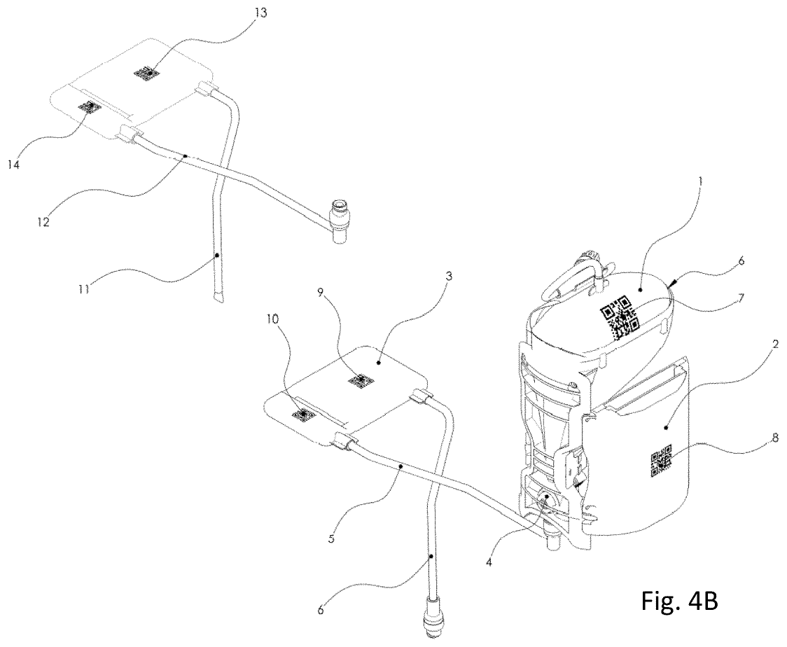

FIG. 3 depicts an embodiment of a cell culture bag (FIGS. 3-1) that is suitable for use with the systems provided by the present disclosure. In the depicted embodiment, the cell culture bag (FIGS. 3-1) has three interlocking ports and connectors (FIGS. 3-3), (FIGS. 3-4), (FIGS. 3-5) to ensure that transfer of the cellular sample into and out of the bag is done within a closed system. The ports and connectors may be spike ports, luer connections or sterile docks for connection using a sterile connection device.

As disclosed herein, the cell culture bag (FIGS. 3-1) includes a unique 2D barcode label (FIGS. 3-6). In some embodiments, the 2D barcode is laser etched onto the bag during processing in order to ensure traceability of the sample back to the subject from which it was derived. In operation, the 2D barcode (FIGS. 3-6) is identical to those that are present on the components of the processing bagset.

In the depicted embodiment, the cell culture bag (FIGS. 3-1) has a cell culture gas-exchange vent (FIGS. 3-2). The vent allows sterile gas exchange in the cell culture bag (FIGS. 3-1). In various embodiments, the gas exchange vent (FIGS. 3-2) is a hydrophobically coated one-way valve that prevents the wetting of vent to allows gas exchange into the bag.

The total volume of cell culture bag may be 50-500 mL, although in several embodiments, when in use it is typically filled with about 200 mL, inclusive of media and sample.

The cell culture bag (FIGS. 3-1) may be a flat bag, having a notched top edge as shown, bottom edge, and two substantially similar side edges. In other embodiments, the top edge may be liner. In the depicted embodiment, the top face of the cell culture bag (FIGS. 3-1) includes the three interlocking ports and connectors (FIGS. 3-3), (FIGS. 3-4), (FIGS. 3-5). The lines of the three ports and connectors may be made of poly(vinyl) chloride (PVC), ethylene-vinyl acetate (EVA), or other materials

The cell culture bag (FIGS. 3-1) may be radio frequency, high frequency or dielectric welded and may be blow-molded.

As noted above, the cell culture bag (FIGS. 3-1) will be placed into an incubator for expansion and/or treatment of the sample. It must thus be made of a material that is capable of withstanding the conditions provided by an incubator.

In various embodiments, the cell culture bag (FIGS. 3-1) may be made of ethylene vinyl acetate (EVA), poly(vinyl) chloride (PVC), polyethylene such as ultra-low density polyethylene, fluorinated ethylene propylene, or other plastics.

### Washing Bagset

FIG. 4A depicts an example of one embodiment of a flexible washing bagset suitable for use with the systems provided by the present disclosure. The depicted flexible bagset comprises a plurality of components. In some embodiments, the components comprise: a processing bag (FIG. 4A-1), cellular waste bag (FIG. 4A-2) and cell concentrate or freezing bag (FIG. 4A-3). Volume transfer between the components is controlled via a multi-port valve (FIG. 4A-4). When an expanded and/or treated cellular sample is loaded into the disclosed systems, it is first loaded into the processing bag (FIG. 4A-1). The multiport valve (FIG. 4A-4) governs the flow of the sample between the other components of the flexible washing bagset.

As disclosed herein, each component of the flexible bagset includes a unique 2D barcode label. In some embodiments, the 2D barcode is laser etched onto each component during processing in order to ensure traceability of the sample back to the subject from which it was derived. All of the 2D barcodes, (FIG. 4A-9), (FIG. 4A-10), (FIG. 4A-11), (FIG. 4A-12), (FIG. 4A-15) and (FIG. 4A-16) are identical. In operation, the 2D barcodes of the washing bagset are identical to those that are present on the components of the processing bagset and the cell culture bag.

The flexible washing bagset is closed to the outside environment and is disposable.

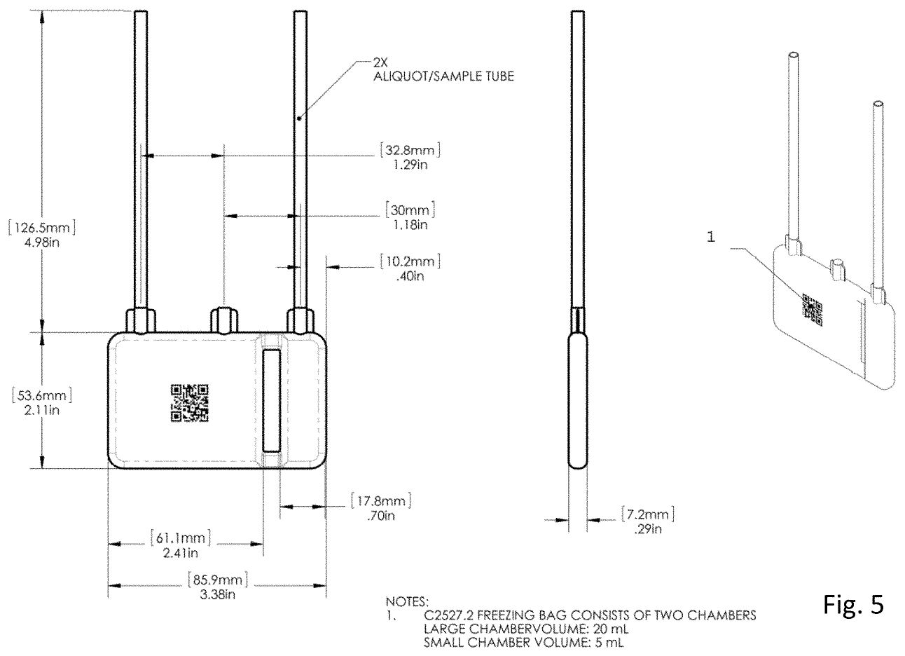

The washing bagset also includes a means for removing the freezing bag (FIG. 4A-3) from the bagset while maintaining the integrity of the closed system. In some embodiments, a removable interlock (FIG. 4A-5) is present to separate the cell concentrate freezing bag from the multiport. In other embodiments, a closed tube path (FIG. 4A-13) from the processing bag (FIG. 4A-1) to the cell concentrate freezing bag (FIG. 4A-3) is present. In each embodiment, the freezing bag (FIG. 4A-3) may be removed from the bagset via the use of luer connections or sterile docks.

As noted above, the bagset comprises a processing bag (FIG. 4A-1), cellular waste bag (FIG. 4A-2) and cell concentrate freezing bag (FIG. 4A-3), all of which are connected by lines or tubing to the multi-port valve (FIG. 4A-4), via inlet lines.

The cell concentrate freezing bag (FIG. 4A-3) further includes a single interlock (FIG. 4A-8) or a sealed extension (FIG. 4A-14) for easy removal of the expanded and/or treated sample from the freezing bag (FIG. 4A-3).

The processing bag (FIG. 4A-1) and the cellular waste bag (FIG. 4A-2) may be made of ethylene vinyl acetate (EVA), poly(vinyl) chloride (PVC) or other plastics. The properties of the freezing bag are described in detail below.

Each of the processing bag (FIG. 4A-1) and the cellular waste bag (FIG. 4A-2) may be blow-molded. In some embodiments, the bags may be radio frequency, high frequency or dielectric welded and may be blow-molded.

In some embodiments, the processing bag (FIG. 4A-1) is a three-dimensional bag having an asymmetric shape, including a top edge, curved side, straight side opposite the curved side, tapered bottom, and bottom outlet that is in fluid connection with the multiport valve (FIG. 4A-4). The top edge includes an inlet for receipt of the expanded and/or processed sample from a cell culture bag, as well as two holes that may be used to hang the processing bag in space. In other embodiments, the processing bag (FIG. 4A-1) may be shaped symmetrically such that its sides taper symmetrically toward the bottom outlet.

The total volume of processing bag may be up to 200 mL, although in several embodiments, when in use it is typically filled with about 50-150 mL of expanded and/or treated cell sample.

In order to receive an expanded and/or treated cell sample, the processing bag (FIG. 4A-1) is configured to receive an inlet line at a discrete point along the top line, which connects to an inlet that is in fluid connection with the interior of the processing bag (FIG. 4A-1). In some embodiments, the inlet line comprises a female luer connection which allows the processing bag set to be connected to a cell culture bag containing the cellular sample to be transferred into processing bag (FIG. 4A-1). In other embodiments, the inlet line comprises a sterile dock for connection to a cell culture bag using a sterile connection device.

The cellular waste bag (FIG. 4A-2) may be a flat bag, having a rounded top edge, bottom edge, and two substantially similar side edges. In some embodiments, the cellular waste bag (FIG. 4A-2) includes a butterfly spike port along the top edge, which can be used to remove an aliquot of the cellular waste products during bio-processing, should that be desired. The bottom edge includes an inlet at one corner that is in fluid connection with the processing bag, via the multiport valve (FIG. 4A-4).

The volume of the cellular waste bag (FIG. 4A-2) is up to 150 mL, although in use it is typically filled with about 30-80 mL.

The lines connecting each of the bags of the washing bagset are tubing that may be made of poly(vinyl) chloride (PVC), ethylene-vinyl acetate (EVA), or other materials.

In some embodiments, multiport valve (FIG. 4A-4) is a three-way stopcock in that has three connectors such that it can be connected to three bags: the processing bag (FIG. 4A-1), cellular waste bag (FIG. 4A-2) and cell concentrate freezing bag (FIG. 4A-3). Other types of metering valves or stopcocks will also work, such as a four-way stopcock having four connectors.

In the depicted embodiment, the multiport valve (FIG. 4A-4) comprises an outer portion having three connectors and an inner portion. The outer portion may be made of polycarbonate. The inner portion includes a handle and barrel, integrally molded, which may be made of polyethylene. The barrel moves between several positions, including a closed position, defined as a position that does not allow any fluid flow through the multiport valve (FIG. 4A-4), and two open positions defined as positions that permit fluid flow through the multiport valve (FIG. 4A-4).

The two open positions include a first position that permits fluid flow from the processing bag (FIG. 4A-1) through the multiport valve (FIG. 4A-4) to the cellular waste bag (FIG. 4A-2) and a second position that permits fluid flow from the processing bag (FIG. 4A-1) through multiport valve (FIG. 4A-4) to the freezing bag (FIG. 4A-3). All fluid flow through the bagset is accomplished via gravity.

FIG. 4B depicts a semi rigid embodiment of the washing bagset suitable for use with the systems provided by the present disclosure. In the depicted embodiment, the semi rigid washing bagset comprises a rigid (ridge) processing bag (FIG. 4B-1), a rigid (ridge) cellular waste container (FIG. 4B-2) and a cell concentrate freezing bag (FIG. 4B-3) for processing a cellular sample. Volume transfer between the bags and container is controlled via a multi-port valve (FIG. 4B-4) when loaded into a CXP-II device. When a cellular sample is loaded into the disclosed systems, it is first loaded into the processing bag (FIG. 4B-1). The multiport valve (FIG. 4B-4) governs the flow of the sample between the other components of the semi rigid processing bagset.

As disclosed herein, each component of the flexible bagset includes a unique 2D barcode label. In some embodiments, the 2D barcode is laser etched onto each component during processing in order to ensure traceability of the sample back to the subject from which it was derived. All of the 2D barcodes, (FIG. 4B-8), (FIG. 4B-9), (FIG. 4B-10), (FIG. 4B-13) and (FIG. 4B-14) are identical. In operation, the 2D barcodes of the washing bagset are identical to those that are present on the components of the processing bagset and the cell culture bag.

The semi rigid washing bagset is closed to the outside environment and is disposable.

The washing bagset also includes a means for removing the freezing bag (FIG. 4B-3) from the bagset while maintaining the integrity of the closed system. In some embodiments, a removable interlock (FIG. 4B-12) is present to separate the cell concentrate freezing bag from the multiport. In other embodiments, a closed tube path (FIG. 4B-5) from the processing bag (FIG. 4B-1) to the cell concentrate freezing bag (FIG. 4B-3) is present. In each embodiment, the freezing bag (FIG. 4B-3) may be removed from the bagset via the use of luer connections or sterile docks.

As noted above, the bagset comprises a processing bag (FIG. 4B-1), a cellular waste container (FIG. 4B-2) and a cell concentrate freezing bag (FIG. 4B-3), all of which are connected by lines or tubing to the multi-port valve (FIG. 4B-4), with inlet lines.

The cell concentrate freezing bag (FIG. 4B-3) further includes a single interlock (FIG. 4B-6) or a sealed extension (FIG. 4B-11) for easy removal of the expanded and/or treated sample from the freezing bag (FIG. 4B-3).

The semi rigid bagset includes a rigid plastic housing for the processing bag (FIG. 4B-1) that maintains its shape and is capable of holding the entire bagset upright during operation. The housing is configured such that it contains a point of attachment for the cellular waste container (FIG. 4B-2), which also includes a rigid plastic backing that connects to the rigid housing. The freezing bag is connected to the processing bag and red blood cell container via a line of tubing and the multiport valve (FIG. 4B-4).

The rigid plastic housing and rigid plastic backing can be made of any suitable rigid-plastic material. The rigid plastic material exhibits no elastic deformation, nor does it display any of the elastic behavior typically displayed by flexible plastics. The rigid plastic material made of any suitable rigid plastic including, for example, high-density polyethylene (HDPE) or polypropylene (PP).

Each of the rigid plastic housing and backing may be injection molded or die-cut.

The processing bag (FIG. 4B-1) is contained within the rigid plastic housing, which may or may not completely enclose the processing bag (FIG. 4B-1). In the depicted embodiment, the processing bag (FIG. 4B-1) is not completely enclosed within the rigid plastic housing; rather, the housing partially encloses the processing bag (FIG. 4B-1), holding it in place during use. The processing bag (FIG. 4B-1) may be made of ethylene vinyl acetate (EVA), poly(vinyl) chloride (PVC) or other plastics. The cellular waste container (FIG. 4B-2) may be made of EVA, PVC or other plastics and is attached to one side of a rigid plastic backing that is removably connected to the rigid plastic housing. The properties of the freezing bag are described in detail below.

Each of the processing bag (FIG. 4B-1) and the cellular waste container (FIG. 4B-2) may be blow-molded. Each may also be radio frequency, high frequency or dielectric welded and may be blow-molded.

In some embodiments, the processing bag (FIG. 4B-1) is a three-dimensional bag having an asymmetric shape, including a top edge, curved side, straight side opposite the curved side, tapered bottom, and bottom outlet that is in fluid connection with the multiport valve (FIG. 4B-4). The top edge includes an inlet for receipt of the sample from a subject, as well as two holes that may be used to hang the processing bag in space. In such embodiments, the shape of the rigid plastic housing mirrors that of the processing bag (FIG. 4B-1), tapering from top to bottom, in order to properly house the processing bag (FIG. 4B-1). In other embodiments, the processing bag (FIG. 4B-1) may be shaped symmetrically such that its sides taper symmetrically towards bottom outlet. In such embodiments, the shape of the rigid plastic housing mirrors that of the processing bag (FIG. 4B-1), having symmetrical sides in order to properly house the processing bag (FIG. 4B-1).

The total volume of processing bag may be up to 200 mL, although in several embodiments, when in use it is typically filled with about 50-150 mL of expanded and/or treated cells from a cell culture bag.

In order to receive the expanded and/or treated sample from a cell culture bag, the processing bag (FIG. 4B-1) is configured to receive an inlet line at a discrete point along the top line, which connects to an inlet that is in fluid connection with the interior of the processing bag (FIG. 4B-1). In some embodiments, the inlet line comprises a female luer connection which allows the processing bag set to be connected to a cell culture bag containing the cellular sample to be transferred into processing bag (FIG. 4B-1). In other embodiments, the inlet line comprises a sterile dock for connection to a cell culture bag using a sterile connection device.

The cellular waste container (FIG. 4B-2) may be a flat bag, having a top edge, bottom edge, and two substantially similar side edges. In some embodiments, the cellular waste container (FIG. 4B-2) includes a butterfly spike port along the top edge, which can be used to remove an aliquot of the cellular waste products during bio-processing, should that be desired. The bottom edge includes an inlet at one corner that is in fluid connection with the processing bag, via the multiport valve (FIG. 4B-4). The container is mounted to the rigid plastic backing that is removably connected to the rigid plastic housing.

The volume of the cellular waste container (FIG. 4B-2) is up to 150 mL, although in use it is typically filled with about 30-80 mL.

The lines connecting each of the bags of the processing bagset are tubing that may be made of poly(vinyl) chloride (PVC), ethylene-vinyl acetate (EVA), or other materials.

In some embodiments, multiport valve (FIG. 4B-4) is a three-way stopcock in that it has three connectors such that it can be connected to three bags: a processing bag (FIG. 4B-1), a cellular waste container (FIG. 4B-2) and a cell concentrate freezing bag (FIG. 4B-3). Other types of metering valves or stopcocks will also work, such as a four-way stopcock having four connectors.

In the depicted embodiment, the multiport valve (FIG. 4B-4) is contained within the rigid plastic housing and comprises an outer portion having three connectors and an inner portion. The outer portion may be made of polycarbonate. The inner portion includes a handle and barrel, integrally molded, which may be made of polyethylene. The barrel moves between several positions, including a closed position, defined as a position that does not allow any fluid flow through the multiport valve (FIG. 4B-4), and two open positions defined as positions that permit fluid flow through the multiport valve (FIG. 4B-4). In other embodiments, the rigid plastic housing may have an opening that is suitable to house the multiport valve (FIG. 4B-4) removably, such that the multiport valve (FIG. 4B-4) may be slid into and out of the housing.

The two open positions include a first position that permits fluid flow from the processing bag (FIG. 4B-1) through the multiport valve (FIG. 4B-4) to the cellular waste container (FIG. 4B-2) and a second position that permits fluid flow from the processing bag (FIG. 4B-1) through multiport valve (FIG. 4B-4) to the freezing bag (FIG. 4B-3). All fluid flow through the bagset is accomplished via gravity.

### Freezing Bag

FIG. 5 depicts an embodiment of a freezing bag suitable for use with the disclosed systems and methods. In the depicted embodiment, the dimensions are approximate.

The freezing bag is a three-dimensional bag that is rectangular in shape, having a top edge, bottom edge, two identical side edges, rounded corners and two internal compartments, a large compartment, and a small compartment—the large and small compartments connected by two channels.

As disclosed herein, the freezing bag includes a unique 2D barcode label (FIGS. 5-1). In some embodiments, the 2D barcode is laser etched onto the freezing bag during processing in order to ensure traceability of the sample back to the subject from which it was derived. In operation, the 2D barcode of the freezing bag is identical to those that are present on the components of the processing bagset, the cell culture bag and the washing bagset.

In some embodiments, the dimensions of the freezing bag are specific and precise, conforming to established standards such as, for example, the CVP.D standard, where V denotes volume, P represents the number of ports leading out of the freezing bag and D represents the depth of the bag, in millimeters. In some embodiments, the freezing bag conforms to the CVP.D standard by having dimensions conforming to the C_{252.72} standard, in which the freezing bag has a total internal storage volume of 25 mL, contains 2 ports, or pigtails, leading out of the freezing bag, and has a thickness depth of 7.2 millimeters (mm). In this embodiment, the large compartment has a total volume of 20 mL and the small compartment has a total volume of 5 mL.

In the depicted embodiment, the top edge of the freezing bag has two ports, or pigtails, leading out of the internal chamber. During bioprocessing, one of the ports may be an inlet that can be connected to the washing bagset via a multiport valve (see, e.g., FIG. 4A). The freezing bag may be removed from the washing bagset in such a way as to maintain the integrity of the closed system, as described above.

Once removed from the washing bagset, the freezing bag can be moved to a cryo storage device. At that time, the two ports can have different functions. For example, the port that is located above the small compartment can be used to remove a sample of the expanded and/or treated cell sample, for quality control purposes. The port located above the large compartment can be used to deliver the contents of the freezing bag back to the subject from which the sample was derived. The ports may be luer connections or sterile docks for connection using a sterile connection device.

The freezing bag is intended for use as a long-term cryo storage device. In that regard, it must be made of components capable of withstanding the extreme low temperatures of cryo preservation. In some embodiments, the freezing bag displays liquid nitrogen stability while remaining impact and puncture resistant.

In some embodiments, the freezing bag is rated for cryogenic preservation of cellular samples, such as peripheral blood for immunotherapy applications, in liquid nitrogen.

In some embodiments, the freezing bag is made from ethylene vinyl acetate (EVA). In some embodiments, the freezing bag is made from a polyolefin-EVA blend. In some embodiments, the freezing bag is made from a fluorinated ethylene propylene (FEP) material, which may conform to USP Class VI standards.

What is claimed is:

1. A system for cellular bioprocessing and manufacturing, comprising: one or more processing bagsets, a culture bagset and a washing bagset, wherein:
each bagset comprises the same 2D barcode that is unique to the system; and
each bagset is configured to be in fluid connection with the other bagsets via one or more luer connections or via one or more sterile docks using a sterile connection device.

2. The system of claim 1, wherein the one or more processing bagsets comprise flexible components.

3. The system of claim 1 or claim 2, wherein the flexible components comprise a processing bag, a red blood cell bag, and a cell concentrate bag;
wherein the system is closed to the outside environment and the components are in fluid connection with each other via a plurality of tubes.

4. The system of claim 3, wherein:
the processing bag is in fluid connection with the red blood cell bag via a first tube;
the processing bag is in fluid connection with the cell concentrate bag via a second tube; and
the red blood cell bag and the cell concentrate bag are not directly connected to each other.

5. The system of claim 3 or claim 4, wherein volume transfer between the components is controlled via a multi-port valve that is directly connected to each of the processing bag, the red blood cell bag, and the cell concentrate bag.

6. The system of any one of claims 2 to 5, wherein the flexible processing bagset is configured for single use and is disposable.

7. The system of any one of claims 2 to 6, wherein:
the processing bag is made from a material selected from ethylene vinyl acetate (EVA), poly(vinyl) chloride (PVC) and other plastics;
the red blood cell bag is made from a material selected from PVC or other plastics; and
the cell concentrate bag is made from EVA, PVC or other plastics.

8. The system of any one of claims 2 to 7, wherein the processing bag comprises an inlet line at the top of the processing bag that is in fluid connection with the interior of the processing bag,
wherein the inlet line comprises a sterile connection selected from a female luer connection and a sterile dock, and
wherein the inlet line is configured for receipt of a sample from outside of the processing bagset.

9. The system of any one of claims 2 to 8, wherein the cell concentrate bag comprises:
a large compartment, and a small compartment connected by two channels; and
one or more ports configured for removal of the contents of the cell concentrate bag away from the processing bagset,
wherein the one or more ports are selected from spike ports, luer connections and sterile docks.

10. The system of claim 5, wherein the multiport valve comprises an outer portion having three connectors and an inner portion, the inner portion comprising a handle and barrel configured to move between a closed position, a first open position and a second open position.

11. The system of claim 10, wherein:
the first open position permits fluid flow from the processing bag through the multiport valve to the red blood cell bag; and
the second position permits fluid flow from the processing bag through multiport valve to the cell concentrate bag.

12. The system of claim 1, wherein the one or more processing bagsets comprise a combination of flexible and rigid components.

13. The system of claim 1 or claim 12, wherein the rigid components comprise a processing container and a red blood cell container, and the flexible components comprise a cell concentrate bag;
wherein the system is closed to the outside environment and the components are in fluid connection with each other via a plurality of tubes.

14. The system of claim 13, wherein:
the processing container is in fluid connection with the red blood cell container via a first tube;
the processing container is in fluid connection with the cell concentrate bag via a second tube; and
the red blood cell container and the cell concentrate bag are not directly connected to each other.

15. The system of claim 13 or claim 14, wherein volume transfer between the components is controlled via a multi-port valve that is directly connected to each of the processing container, the red blood cell container, and the cell concentrate bag.

16. The system of any one of claims 12 to 15, wherein the processing bagset is configured for single use and is disposable.

17. The system of any one of claims 12 to 16, wherein:
the processing container is made from a material selected from ethylene vinyl acetate (EVA), poly(vinyl) chloride (PVC) and other plastics;
the red blood cell container is made from a material selected from PVC or other plastics; and
the cell concentrate bag is made from EVA, PVC, or other plastics.

18. The system of any one of claims 12 to 17, wherein the processing container comprises an inlet line at the top of the processing container that is in fluid connection with the interior of the processing container,
wherein the inlet line comprises a sterile connection selected from a female luer connection and a sterile dock, and
wherein the inlet line is configured for receipt of a sample from outside of the processing bagset.

19. The system of any one of claims 12 to 18, wherein the cell concentrate bag comprises:
a large compartment, and a small compartment connected by two channels; and
one or more ports configured for removal of the contents of the cell concentrate bag away from the processing bagset,
wherein the one or more ports are selected from spike ports, luer connections and sterile docks.

20. The system of claim 15, wherein the multiport valve comprises an outer portion having three connectors and an inner portion, the inner portion comprising a handle and barrel configured to move between a closed position, a first open position and a second open position.

21. The system of claim 20, wherein:
the first open position permits fluid flow from the processing container through the multiport valve to the red blood cell container; and
the second position permits fluid flow from the processing container through multiport valve to the cell concentrate bag.

22. A three-dimensional freezing bag, comprising:
an interior chamber, comprising a large compartment and a small compartment, the compartments connected by two channels;
a first port defining a fluid connection between the large compartment and the exterior of the freezing bag;
a second port defining a fluid connection between the small compartment and the exterior of the freezing bag; and
a unique 2D barcode label;
wherein the freezing bag is constructed for long-term cryo storage.

23. The freezing bag of claim 22, wherein the freezing bag conforms to the C_{252.72} standard, wherein:
the internal volume of the storage bag is 25 mL,
there are a total of 2 ports leading out of the interior chamber of the freezing bag, and
the freezing bag has a thickness depth of 7.2 mm.

24. The freezing bag of claim 22 or claim 23, wherein the large compartment has a total volume of 20 mL and the small compartment has a total volume of 5 mL.

25. The freezing bag of any one of claims 22 to 24, wherein:
the first port is configured to receive a cellular sample from outside of the freezing bag, and
the first port is also configured to deliver the contents of the large chamber outside of the freezing bag.

26. The freezing bag of any one of claims 22 to 25, wherein:
the second port is configured to deliver at least some of the contents of the small compartment outside of the freezing bag.

27. The freezing bag of any one of claims 22 to 26, wherein the ports comprise sterile connections selected from luer connections and sterile docks for connection using a sterile connection device.

28. The freezing bag of any one of claims 22 to 27, wherein the freezing bag is rated for cryogenic preservation of cellular samples in liquid nitrogen.

29. The freezing bag of any one of claims 22 to 28, wherein the freezing bag is made from a material selected from ethylene vinyl acetate (EVA), a polyolefin-EVA blend, a fluorinated ethylene propylene (FEP) material, and combinations of any of the foregoing.

30. The freezing bag of any one of claims 12 to 19, wherein the unique 2D barcode corresponds to a 1D barcode present on a cryogenic storage cassette.

31. A method of producing and cryo storing an engineered autologous cellular product, comprising:
obtaining a cellular sample from a subject;
transferring the sample to one or more cell processing bagsets without exposing the sample to the outside environment, by attaching male and female luer lock connectors between the container in which the sample is obtained and the one or more processing bagsets, or by sterile-docking the tubing of the container in which the sample was obtained and the one or more processing bagsets using a sterile connection device;
placing the one or more processing bagsets in one or more processing containers;
centrifuging the one or more processing containers, thereby stratifying and separating the cellular sample based on the density, size of the cells and starting volume;
transferring the desired cellular concentrate via gravity flow from the one or more processing bagsets to a culture bag without exposing the sample to the outside environment, by attaching male and female luer lock connectors between the one or more processing bagsets and the culture bag, or by sterile-docking the tubing of the one or more processing bagsets and the tubing of the culture bag using a sterile connection device;
incubating the culture bag, thereby expanding the desired cellular concentrate;
transferring the expanded cellular concentrate via gravity flow from the culture bag to a washing bagset which, by attaching male and female luer lock connectors to each other between the culture bag and the washing bagset, or sterile-docking the tubing of the culture bag and the washing bagset using a sterile connection device;
washing the expanded cellular concentrate, thereby separating cellular waste byproducts generated from expansion from an engineered cell product;
transferring the engineered cell product from the washing bagset to a freezing bag by attaching male and female luer lock connectors to each other between the washing bagset and the freezing bag, or sterile-docking the tubing of the washing bagset and the freezing bag using a sterile connection device, wherein the transfer occurs via centifugal force or gravity flow;
transferring the freezing bag into a cryo-freezing overwrap bag and canister; and
transferring the freezing bag, cryo-freezing overwrap and canister into a controlled rate cryo-freezing system that uses liquid nitrogen vapor to freeze the engineered cell product and maintain it in a cryo-preserved state.

32. The method of claim 31, wherein the cellular sample is selected from peripheral blood, whole blood, bone marrow, cord blood, and combinations of any of the foregoing.

33. The method of claim 31 or claim 32, wherein the one or more cell processing bagsets, the culture bagset, the washing bagset and the freezing bag:
are all configured for single use;
are disposable; and
all comprise the same unique 2D barcode that is specific to the sample.

34. The method of any one of claims 31 to 33 further comprising, prior to centrifugation, depleting red blood cells from the sample.

35. The method of any one of claims 31 to 34, wherein the sample is peripheral blood, red blood cells are depleted from the sample prior to centrifugation, and the centrifugation separates the peripheral blood into red blood cells, stem cell fraction and plasma.

36. The method of any one of claims 31 to 35, wherein, prior to the incubation of the culture bag, supplementing a cellular growth media contained within the culture bag with one or more additives selected from cytokines, glucose and both.

37. The method of any one of claims 31 to 36, wherein the freezing bag is compliant with C_{252.72} standards, having a 25 milliliter (mL) storage volume, 2 ports or pig tails, and a depth of 7.2 mm.

38. The method of any one of claims 31 to 37, wherein the canister comprises a unique 1D barcode that is coupled to the 2D barcode on the freezing bag.

39. The method of claim 38, further comprising, during the transfer of the freezing bag, cryo-freezing overwrap and canister into a controlled rate cryo-freezing system, scanning the 1D barcode to confirm the coupling of the 1D barcode information to the 2D barcode information.

40. The method of any one of claims 31 to 39, further comprising, after the transfer to the cryo-freezing system, transferring the freezing bag, cryo-freezing overwrap and canister to a storage location in a flask filled with liquid nitrogen for long-term cryo-storage.
